(19) United States
(12) Patent Application Publication
(10) Pub. No.: US 2022/0395620 A1
(43) Pub. Date: Dec. 15, 2022

(54) PLASMA SEPARATION DEVICES AND RELATED METHODS

(71) Applicant: Tasso, Inc., Seattle, WA (US)

(72) Inventors: Bryce Peterson, Seattle, WA (US); Emily Welch, Seattle, WA (US); Erwin Berthier, Seattle, WA (US); Ben Casavant, Seattle, WA (US)

(21) Appl. No.: 17/769,803

(22) PCT Filed: Oct. 16, 2020
(86) PCT No.: PCT/US2020/055916

§ 371 Date: Apr. 18, 2022

Related U.S. Application Data
(60) Provisional application No. 62/923,379, filed on Oct. 18, 2019.

(51) Int. Cl. A61M 1/36

(52) U.S. Cl. CPC A61M 1/3633; A61M 1/3693; A61M 2202/0415

(57) ABSTRACT

Devices and methods for withdrawing blood from a patient and separating plasma from the blood are disclosed herein. In some embodiments, a fluid cartridge can be coupled to a collection device and configured to receive whole blood withdrawn from the patient using the collection device. The cartridge can include a housing having a reservoir portion configured to receive the whole blood, and a plasma separation substrate positioned within the housing. The substrate can be folded to define (a) a crease, (b) a first strip portion extending away from the crease, and (c) a second strip portion extending away from the crease. The crease can be positioned adjacent to the reservoir portion to receive the whole blood, and the plasma separation substrate can be configured to wick the whole blood along the first and second strip portions to separate the plasma from the whole blood.

### CROSS-REFERENCE TO RELATED APPLICATION

This application claims the benefit of U.S. Provisional Patent Application No. 62/923,379, filed Oct. 18, 2019, and titled “PLASMA SEPARATION DEVICES AND RELATED METHODS,” which is incorporated herein by reference in its entirety.

### TECHNICAL FIELD

The present technology is related to the collection of bodily fluids and their delivery and storage in removable devices, such as dried plasma separation cartridges.

### BACKGROUND

Devices, systems, and methods to collect bodily fluids are necessary devices for the growing field of personalized medicine. While analysis laboratories are well suited to perform diagnostic tests, the collection of blood samples remains challenging, in particular for patients that do not have simple access to a blood testing laboratory. These patients can be located in rural areas, underserved sub-urban areas, or low resource areas and have significant barriers to accessing diagnostic services. To reach patients in any location and connect them with blood testing facilities, robust systems for sample encapsulation, stabilization, and shipping must be developed.

### BRIEF DESCRIPTION OF THE DRAWINGS

Many aspects of the present technology can be better understood with reference to the following drawings. The components in the drawings are not necessarily to scale. Instead, emphasis is placed on clearly illustrating the principles of the present technology.

FIG. 1A is a perspective view of a bodily fluid collection device configured in accordance with an embodiment of the present technology; FIG. 1B is a perspective view of the bodily fluid collection device during use; and FIG. 1C is a perspective view illustrating detachment of a collection cartridge from the bodily fluid collection device.

FIGS. 2A-2C are an exploded isometric view, a rear isometric view, and a side cross-sectional view, respectively, of the collection cartridge of FIGS. 1A-1C configured in accordance with an embodiment of the present technology.

FIG. 3 is a side cross-sectional view of the collection cartridge configured in accordance with another embodiment of the present technology.

FIG. 4 is an exploded isometric view of a collection cartridge configured in accordance with another embodiment of the present technology.

FIGS. 5A and 5B are an exploded isometric view and a front view, respectively, of a collection cartridge configured in accordance with another embodiment of the present technology.

FIGS. 6A-6C are an isometric front view, a cross-sectional side view, and a front view, respectively, of a collection cartridge configured in accordance with another embodiment of the present technology.

FIGS. 7A and 7B are an exploded isometric view and a front view, respectively, of a collection cartridge configured in accordance with another embodiment of the present technology.

FIGS. 8A and 8B are side cross-sectional views of a collection cartridge in a first position and a second position, respectively, and configured in accordance with another embodiment of the present technology.

FIGS. 9A and 9B are side cross-sectional views of a collection cartridge in a first position and a second position, respectively, and configured in accordance with another embodiment of the present technology.

FIG. 10 is a schematic view of a portion of a collection cartridge configured in accordance with another embodiment of the present technology.

FIGS. 11A-11C are an isometric view, a side view, and a top view, respectively, of a collection cartridge configured in accordance with another embodiment of the present technology.

FIGS. 12A and 12B are a side view and a top view, respectively, of a collection cartridge configured in accordance with another embodiment of the present technology.

FIG. 13A is an enlarged isometric view of a collection cartridge configured in accordance with an embodiment of the present technology.

FIG. 13B is an enlarged isometric view of an interior portion of the bodily fluid collection device shown in FIGS. 1A-1C configured in accordance with an embodiment of the present technology.

FIGS. 13C and 13D are enlarged isometric view of the collection cartridge of FIG. 13A coupled to the portion of the bodily fluid collection device shown in FIG. 13B in a first position and a second position, respectively, in accordance embodiments of the present technology.

### DETAILED DESCRIPTION

The present technology is directed generally to devices and methods for withdrawing and collecting whole blood from a patient, and separating plasma from the whole blood. In some embodiments, a fluid cartridge can be coupled to a collection device and configured to receive whole blood withdrawn from the patient using the collection device. The cartridge can include a housing having a reservoir portion configured to receive the whole blood, and a plasma separation substrate (e.g., a strip of plasma separation paper) positioned within the housing. The plasma separation substrate can be folded to define a crease, a first strip portion extending away from the crease, and a second strip portion extending away from the crease. The crease can be positioned adjacent to the reservoir portion to receive the whole blood. The plasma separation substrate can be configured to wick the whole blood along the first and second strip portions away from the crease to separate the plasma from the whole blood. In one aspect of the present technology, the folded configuration of the plasma separation substrate is expected to provide a larger plasma yield per volume of whole blood than a conventional arrangement (e.g., a planar or linear arrangement) in which bodily fluid enters the plasma separation substrate through a first end and then wicks along the entire length of the plasma separation substrate.

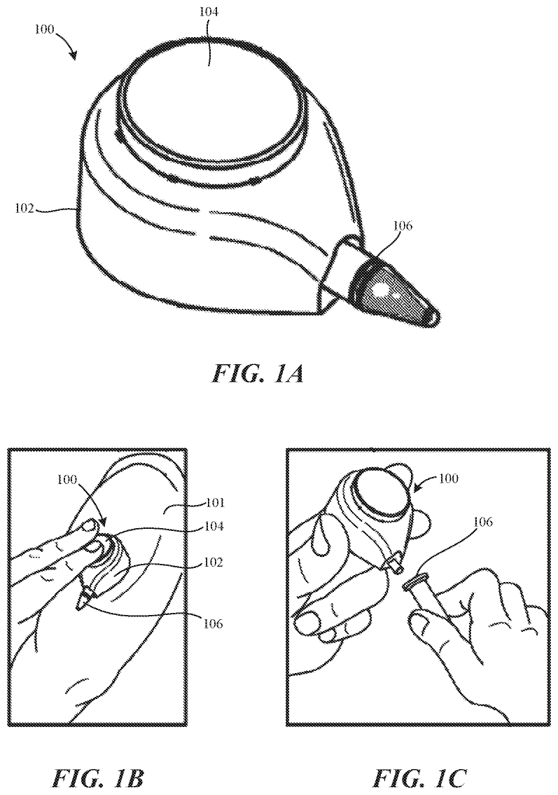

Specific details of several embodiments of the present technology are described herein with reference to FIGS. 1A-10. However, the present technology may be practiced without some of these specific details. In some instances, well-known structures and techniques often associated with bodily fluid collection devices, plasma separation substrates and techniques, and the like, have not been shown in detail so as not to obscure the present technology. The terminology used in the description presented below is intended to be interpreted in its broadest reasonable manner, even though it is being used in conjunction with a detailed description of certain specific embodiments of the disclosure. Certain terms may even be emphasized below; however, any terminology intended to be interpreted in any restricted manner will be overtly and specifically defined as such in this Detailed Description section.

The accompanying Figures depict embodiments of the present technology and are not intended to be limiting of its scope. The sizes of various depicted elements are not necessarily drawn to scale, and these various elements may be arbitrarily enlarged to improve legibility. Component details may be abstracted in the Figures to exclude details such as position of components and certain precise connections between such components when such details are unnecessary for a complete understanding of how to make and use the present technology. Many of the details, dimensions, angles, and other features shown in the Figures are merely illustrative of particular embodiments of the disclosure. Accordingly, other embodiments can have other details, dimensions, angles, and features without departing from the spirit or scope of the present technology.

FIG. 1A is a perspective view of a bodily fluid collection device 100 (“device 100”) configured in accordance with an embodiment of the present technology. The device 100 can be handheld with a size that is easily grasped and manipulated by one or both of a patient's hands. Such handheld devices advantageously allow a patient to collect a bodily fluid sample (e.g., a blood sample) without assistance from another individual. In some embodiments, the handheld devices of the present technology can be operated by a layperson outside of a medical setting (e.g., at home or in a field clinic) and without the aid of a medical professional.

In the illustrated embodiment, the device 100 includes a housing 102 and an actuator 104. The actuator 104 (e.g., a button) can be movable relative to the housing 102 to actuate/initiate withdrawal of a bodily fluid from the patient. The housing 102 is removably coupled to a collection cartridge 106 (e.g., a tube, reservoir) for receiving the bodily fluid withdrawn from the patient. The cartridge 106 can act as a removable and standardized container for bodily fluid that can be detached and used in clinical and laboratory equipment or workflows (e.g., for diagnostics and/or biomarker detection).

FIG. 1B is a perspective view of the bodily fluid collection device 100 in use by the patient. To collect a bodily fluid sample, the device 100 is applied to a patient's body, with a bottom surface of the housing 102 positioned against the skin 101 of the patient and the actuator 104 positioned away from the skin 101. Actuating (e.g., pressing, twisting, pulling) the actuator 104 deploys a skin-piercing feature (e.g., a blade, lancet) from within the device 100 to pierce the skin 101. In some embodiments, the device 100 is configured to generate a vacuum within the device 100 that acts against the patient's skin either directly or indirectly, and before and/or after deployment of the skin-piercing feature. Bodily fluid from the resulting incision is withdrawn into the housing 102 and collected into the cartridge 106.

FIG. 1C is a perspective view illustrating detachment of the cartridge 106 from the device 100. Once the desired amount of bodily fluid has been collected into the cartridge 106, the device 100 is removed from the skin 101, and the cartridge 106 is detached from the housing 102.

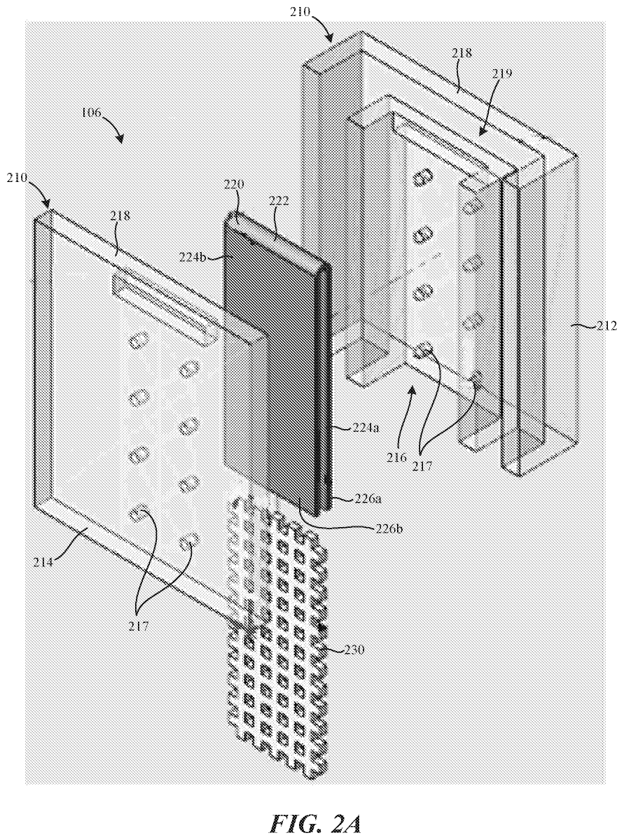

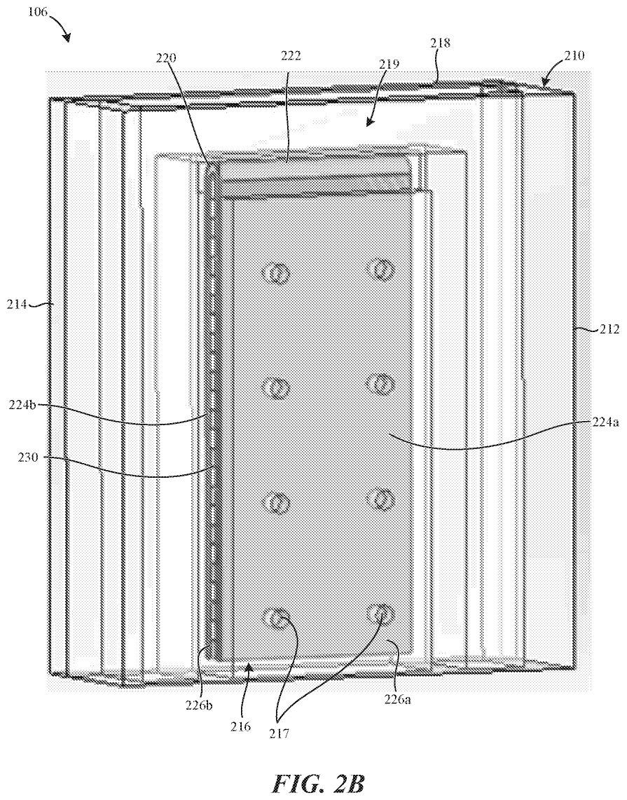

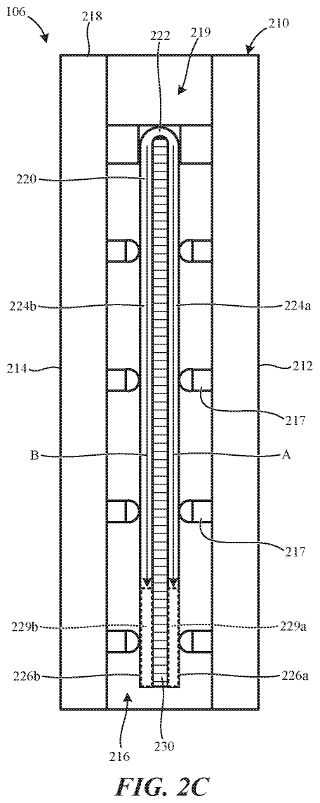

FIGS. 2A-2C are an exploded isometric view, a rear isometric view, and a side cross-sectional view, respectively, of the cartridge 106 configured in accordance with an embodiment of the present technology. Referring to FIGS. 2A-2C together, the cartridge 106 includes a housing 210, a substrate 220, and a divider 230. The housing 210 is shown as partially transparent in FIGS. 2A and 2B for of clarity.

In the illustrated embodiment, the housing 210 includes a base 212 defining a chamber/channel 216, and a cover 214 configured to be coupled to the base 212 to at least partially enclose the channel 216. In some embodiments, the cover 214 can be removably coupled to the base 212 to, for example, permit removal of the substrate 220 as described in detail below. In other embodiments, the cover 214 can be permanently attached to the base 212, or the cover 214 and the base 212 can be integrally formed together.

The housing 210 further includes an upper portion 218 configured to be removably coupled to the device 100 (FIGS. 1A-1C). In some embodiments, the upper portion 218 can include one or more mating features (e.g., flanges), connectors, tubes, and the like (not shown) for coupling the cartridge 106 to the device 100. The housing 210 further defines a reservoir portion 219 adjacent to (e.g., above) the channel 216. The housing 210 may be composed of metal, plastic, and/or other suitable materials. For example, in some embodiments the housing 210 can be 3D-printed, molded (e.g., injection molded), or otherwise formed from a plastic material.

The substrate 220 can be positioned at least partially around the divider 230 and in the channel 216 of the housing 210. More specifically, in the illustrated embodiment the substrate 220 is folded (e.g., around the divider 230) to define (i) a crease 222, (ii) a first strip portion 224a extending away from the crease 222, and (iii) a second strip portion 224b extending away from the crease 222. As best seen in FIGS. 2B and 2C, the substrate 220 is positioned within the channel 216 such that the crease 222 is positioned proximate to the reservoir portion 219 of the housing 210. In some embodiments, the crease 222 can act as a lower surface of the reservoir portion 219 and/or can project/extend into the reservoir portion 219. In the illustrated embodiment, the first and second strip portions 224a, b have generally the same length (e.g., the substrate 220 is folded generally in half). Further, the first and second strip portions 224a, b extend generally parallel to each other. In other embodiments, however, the substrate 220 can be folded differently such that the first and second strip portions 224a, b have different arrangements relative to each other and/or different relative lengths. Moreover, in the illustrated embodiment the housing 210 includes a plurality of stand-off members 217 projecting inward from the cover 214 and the base 212 and toward the substrate 220. The stand-off members 217 (e.g., tabs, projections) can engage/contact the substrate 220 to secure/suspend the substrate 220 within the channel 216 of the housing 210. In one aspect of the present technology, the stand-off members 217 secure the substrate 220 in the channel 216 while contacting only a relatively small area of the substrate 220.

The substrate 220 may be composed of any suitable material for receiving, collecting, and/or stabilizing bodily fluid, such as a paper or fiber matrix. In some embodiments, the substrate 220 is configured to separate plasma from a sample of whole blood. For example, the substrate 220 can comprise a strip of plasma separation paper that preferentially allows plasma to flow from the crease 222 and along/through (i) the first strip portion 224a toward a first end portion 226a of the substrate 220 and (ii) the second strip portion 224b toward a second end portion 226b of the substrate 220. In other embodiments, the substrate 220 can include two or more different types of substrates. For example, the substrate 220 (e.g., the first and/or second strip portions 224a, b) could include a first substrate configured to receive and store whole blood (or various constituents thereof), and a second substrate adjacent the first substrate and configured to separate plasma from the whole blood. As one of ordinary skill in the art will appreciate, a myriad of combinations of different substrates can be used and are within the scope of the present technology.

In the illustrated embodiment, the divider 230 is a mesh (e.g., a plastic mesh) positioned between the first and second strip portions 224a, b of the substrate 220 to maintain separation therebetween. The divider 230 can inhibit or even prevent fluid flow thereacross between the first and second strip portions 224a, b. In other embodiments, the divider 230 can have a different configuration/arrangement (e.g., be a solid member), or the divider 230 can be omitted.

In operation, bodily fluid (e.g., whole blood) is received in the reservoir portion 219 where it may begin to pool. For example, referring to FIGS. 1A-2C together, the patient can actuate the actuator 104 to deploy the skin-piercing feature from within the device 100 to pierce the skin 101 to draw blood, and the device 100 can direct the blood (e.g., via a microfluidic or other fluidic network, gravitational force, capillary force) into the cartridge 106. The blood then flows from the reservoir portion 219 into the substrate 220. More specifically, in the illustrated embodiment the blood can flow from the reservoir portion 219 into the crease 222 and then wick along/through both (i) the first strip portion 224a toward the first end portion 226a (e.g., as indicated by arrow A in FIG. 2C) and/or (ii) the second strip portion 224b toward the second end portion 226b (e.g., as indicated by arrow B in FIG. 2C). The divider 230 can inhibit the blood from wicking/flowing between (e.g., laterally between) the first and second strip portions 224a, b.

In some embodiments, the substrate 220 can act to separate plasma from the whole blood as the blood is wicked along the first and second strip portions 224a, b. For example, the substrate 220 can be configured to promote a differential flow rate between the plasma and other blood constituents such that plasma is separated and collected farther toward the first and second end portions 226a, b of the substrate 220. For example, plasma can be substantially separated and collected in/along a first plasma portion 229a (shown in dashed lines in FIG. 2C) of the first strip portion 224a and a second plasma portion 229b (shown in dashed lines in FIG. 2C) of the second strip portion 224b, while other constituents of the blood are collected in/along the rest of the substrate 220.

In some embodiments, the housing 210 can include holes, openings, or other features to facilitate drying of the substrate 220 while it remains within the housing 210. In some embodiments, the cartridge 106 can be separated from the device 100 to allow the blood/plasma to dry in the substrate 220 and to facilitate testing thereof. For example, the cover 214 can be removed from the base 212 to allow access to the substrate 220 for testing after collecting bodily fluid in the substrate 220. In some embodiments, the substrate 220 can include perforations to facilitate separation of one or more portions of the substrate 220 for testing. For example, in some embodiments the first and second plasma portions 229a, b can be removed (e.g., torn off) to facilitate testing of the plasma stored therein. In other embodiments, the cartridge 106 can be configured such that one or more portions of the substrate 220 can be “punched out” or otherwise removed for testing. For example, the housing 210 can include one or more windows/opening formed in the cover 214 and/or the base 212 (not shown) to allow portion(s) of the substrate 220 beneath the window(s) to be removed.

In one aspect of the present technology, the folded configuration of the substrate 220 is expected to provide a better (e.g., larger) plasma yield than conventional arrangements of plasma separation substrates, such as planar or linear arrangements in which bodily fluid enters a substrate through an end of the substrate and then wicks along the entire length of the substrate. More specifically, it is expected that the folded configuration of the substrate 220 can increase the chromatographic effect of the substrate 220. Further, the stand-off members 217 can help increase wicking of bodily fluid along the substrate 220 and/or plasma separation by positioning/suspending the substrate 220 away from the rest of the housing 210 and contacting only a relatively small area of the substrate 220.

In some embodiments, the cartridge 106 can include features for inhibiting or even preventing oversaturation of the substrate 220 which may cause, for example, other blood constituents to contaminate the first and second plasma portions 229a, b. For example, FIG. 3 is a side cross-sectional view of the cartridge 106 configured in accordance with another embodiment of the present technology and including a pair of separation buttons 340 (identified individually as a first separation button 340a and a second separation button 340b). In the illustrated embodiment, each of the separation buttons 340 includes a substrate-piercing feature 342 (e.g., a blade, lancet) configured to pierce, cut, and/or separate a portion of the substrate 220. More specifically, the first separation button 340a is positioned along the base 212 of the housing 210 with the substrate-piercing feature 342 aligned with the start of the first plasma portion 229a of the substrate 220, and the second separation button 340b is positioned along the cover 214 of the housing 210 with the substrate-piercing feature 342 aligned with the start of the second plasma portion 229b of the substrate 220.

In operation, the patient can actuate (e.g., depress, twist) one or both of the separation buttons 340 inward toward the substrate 220 to separate one or both of the first and second plasma portions 229a, b from the rest of the substrate 220. For example, the patient can actuate the separation buttons 340 after a predetermined time at which it is expected that the first and second plasma portions 229a, b will be saturated with plasma. This can fluidly disconnect the first and second plasma portions 229a, b from the rest of the substrate 220 such that additional blood (or other bodily fluid) wicking along the substrate 220 from the reservoir portion 219 will not saturate and potentially contaminate the first and second plasma portions 229a, b. In other embodiments, the cartridge 106 can include only a single separation button configured to separate both the first and second strip portions 224a, b of the substrate 220 (e.g., and the divider 230), or can include a greater number of separation buttons configured to separate the substrate 220 at multiple locations. In some embodiments, the separation buttons 340 can include locking features to inhibit their accidental deployment.

In some embodiments, the cartridge 106 can alternatively or additionally include one or more overflow substrates 350 (identified individually as first through fifth overflow substrates 350a-350e, respectively) positioned within the housing 210 and configured to receive/direct excess bodily fluid away from the substrate 220 (e.g., away from the first and/or second plasma portions 229a, b). The overflow substrates 350 can be sponges, high-absorbance papers, or the like configured to collect excess fluid above a saturation capacity/volume of the substrate 220. In some embodiments, the overflow substrates 350 can individually or collectively have saturation volumes that are greater than the saturation volume of the substrate 220. The overflow substrates 350 can have any suitable shape and can be placed anywhere within the housing 210. In the illustrated embodiment, for example, (i) the first overflow substrate 350a is positioned within the reservoir portion 219, (ii) the second and third overflow substrates 350b, c are positioned adjacent the substrate 220 within the channel 216 and proximate to the crease 222, and (iii) the fourth and fifth overflow substrates 350d, e are positioned adjacent the substrate 220 within the channel 216 and proximate to the first and second plasma portions 229a, b, respectively. In other embodiments, one or more of the overflow substrates 350 can be incorporated into/positioned within the divider 230. As one of ordinary skill in the art will appreciate, a myriad of combinations of the overflow substrates 350 are possible and within the scope of the present technology.

In yet other embodiments, during operation, the patient can simply disconnect the cartridge 106 from the device 100 (FIGS. 1A-1C) after a predetermined time to inhibit oversaturation of the substrate 220. In some embodiments, the cartridge 106 can include a fill indicator (e.g., a transparent window including tick/hash marks) configured to signal to the user when an appropriate volume of blood has been collected.

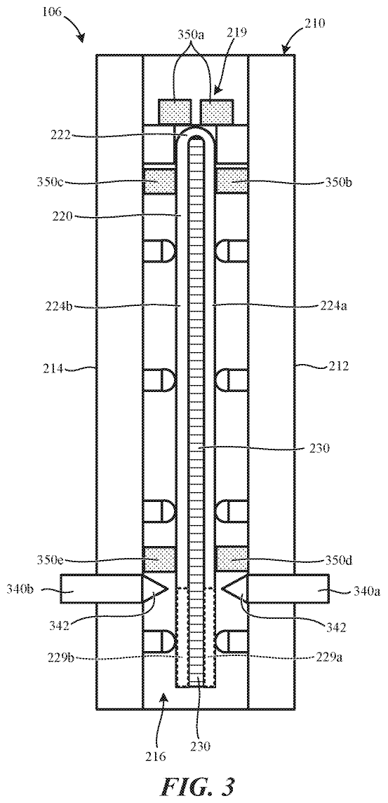

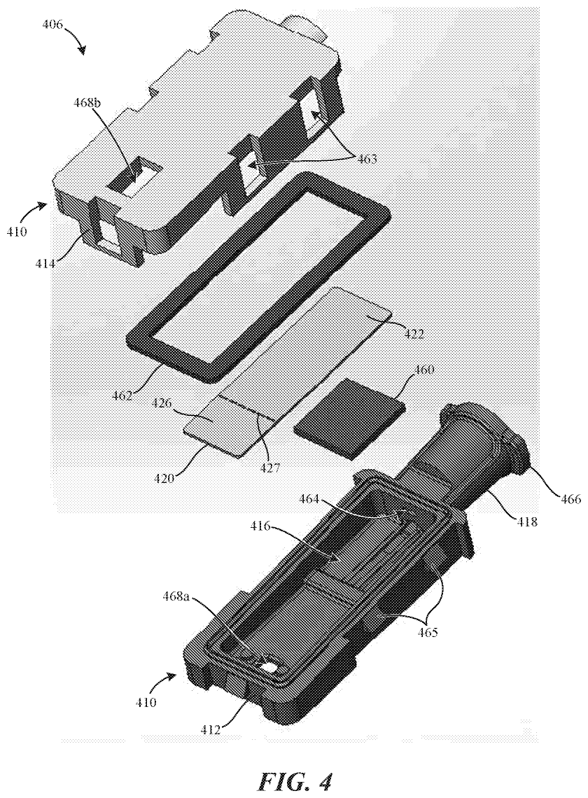

FIG. 4 is an exploded isometric view of a collection cartridge 406 configured in accordance with another embodiment of the present technology. The cartridge 406 can include some features generally similar or identical to the features of the cartridge 106 described in detail above with reference to FIGS. 1A-3, and can be used with the bodily fluid collection device 100 instead of or in addition to the cartridge 106.

In the illustrated embodiment, the cartridge 406 includes a housing 410, a first substrate 420, a second substrate 460, and a sealing member 462. The housing 410 can include a base 412 defining a chamber/channel 416, and a cover 414 configured to be coupled to the base 412 to at least partially enclose the channel 416. In some embodiments, the cover 414 can be removably coupled to the base 412 via, for example, a snap-fit arrangement between a plurality of retaining portions 463 formed in the cover 414 and corresponding tabs/projections 465 formed on the base 412. In one aspect of the present technology, this arrangement can permit the first substrate 420 and/or the second substrate 460 to be removed from the housing 410 (e.g., for testing). In other embodiments, the cover 414 can be permanently attached to the base 412, or the cover 414 and the base 412 can be integrally formed together. In the illustrated embodiment, the base 412 further includes an upper portion 418 (e.g., a coupling portion) configured to be removably coupled to the device 100 (FIGS. 1A-1C). The upper portion 418 can define an inflow channel 464 for receiving a flow of bodily fluid (e.g., blood), and can include one or more mating features 466 (e.g., flanges) for releasably coupling the cartridge 406 to the device 100. The sealing member 462 is configured to seal an interface between the base 412 and the cover 414. In some embodiments, the sealing member 462 can be omitted.

The first and second substrates 420, 460 may be composed of any suitable material for receiving, collecting, and/or stabilizing bodily fluid, such as a paper or fiber matrix. In some embodiments, the first substrate 420 is configured to separate plasma from a sample of whole blood. For example, the first substrate 420 can comprise a strip of plasma separation paper that preferentially allows plasma to flow from a first end portion 422 to a second end portion 426 (e.g., a plasma collection portion) of the first substrate 420. In some embodiments, the second substrate 460 can be thicker and/or can be relatively more absorbent than the first substrate 420. In the illustrated embodiment, the second substrate 460 is positioned within the channel 416 and proximate the inflow channel 464, and the first substrate 420 is positioned within the base 412 on (e.g., in contact with) the second substrate 460.

In operation, bodily fluid (e.g., whole blood) is received in the channel 416 via the inflow channel 464 (e.g., after the patient actuates the actuator 104 to deploy the skin-piercing feature from within the device 100, as shown in FIGS. 1A-1C). The blood initially flows from the inflow channel 464 into the second substrate 460. The second substrate 460 then transfers the blood into the first end portion 422 of the first substrate 420, and the first substrate 420 wicks the blood from the first end portion 422 to the second end portion 426 such that plasma is separated and collected farther toward the second end portion 426 of the first substrate 420. In one aspect of the present technology, the second substrate 460 can quickly absorb a large volume of the blood and then transfer the blood to the first substrate 420 at a controlled rate. In some embodiments, an absorbency, size, volume, and/or other characteristic of the second substrate 460 can be selected to achieve a desired flow/transfer rate from the second substrate 460 to the first substrate 420.

The cartridge 400 is configured to allow the removal a portion of the first substrate 420 for testing. More specifically, in the illustrated embodiment (i) the first substrate 420 includes perforations 427 proximate the second end portion 426, (ii) the base 412 includes a first aperture 468a, and (iii) the cover 414 includes a second aperture 468b. The first and second apertures 468a, b are aligned with one another and with the second end portion 426 of the first substrate 420. In operation, the second end portion 426 (e.g., containing separated plasma) can be removed (e.g., punched out) from within the housing 410 via the first and second apertures 468a, b. More specifically, for example, an operator at a testing facility can insert a probe into the first aperture 468a and against the second end portion 426 of the first substrate 420 to (i) separate the first substrate 420 along the perforations 427 and (ii) force the separated second end portion 426 out of the cartridge 406 via the first aperture 468a in the base 412.

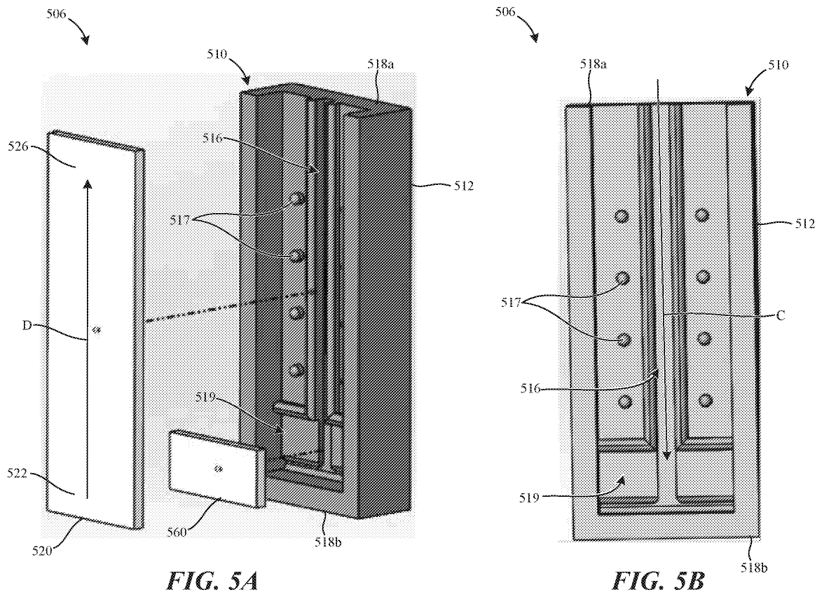

FIGS. 5A and 5B are an exploded isometric view and a front view, respectively, of a collection cartridge 506 configured in accordance with another embodiment of the present technology. Referring to FIGS. 5A and 5B together, the cartridge 506 can include some features generally similar or identical to the features of the cartridges 106 and 406 described in detail above with reference to FIGS. 1A-4, and can be used with the bodily fluid collection device 100.

In the illustrated embodiment, the cartridge 506 includes a housing 510, a first substrate 520, and a second substrate 560. The first and second substrates 520, 560 are not shown in FIG. 5B for clarity. The housing 510 includes a base 512 including an upper portion 518a and a lower portion 518b (e.g., a base portion). The upper portion 518a is configured to be removably coupled to the device 100 (FIGS. 1A-1C) and, in some embodiments, can include one or more mating features (e.g., flanges), connectors, tubes, and/or features (not shown) for coupling the cartridge 506 to the device 100. In the illustrated embodiment, the housing 510 defines (i) a channel 516 extending from the upper portion 518a toward the lower portion 518b of the base 512, and (ii) a reservoir portion 519 proximate the lower portion 518b of the base 512 and fluidly connected to the channel 516. In some embodiments, the housing 510 can further include a cover (not shown) configured to be coupled to the base 512 to cover and/or seal the channel 516 and/or the reservoir portion 519.

The first and second substrates 520, 560 may be composed of any suitable material for receiving, collecting, and/or stabilizing bodily fluid, such as a paper or fiber matrix. In some embodiments, the first substrate 520 is configured to separate plasma from a sample of whole blood, while the second substrate 560 can be thicker and/or relatively more absorbent than the first substrate 520 for quickly absorbing and transferring bodily fluid to the first substrate 520. In the illustrated embodiment, the second substrate 560 is positioned within the reservoir portion 519 of the base 512, and the first substrate 520 is positioned within the base 512 and in fluid communication with (e.g., contacting) the second substrate 560. Moreover, in the illustrated embodiment the housing 510 includes a plurality of stand-off members 517 projecting inward from the base 512 and toward the first substrate 520. The stand-off members 517 (e.g., tabs, projections) can engage/contact the first substrate 520 to secure/suspend the first substrate 520 within the base 512. In one aspect of the present technology, the stand-off members 517 secure the first substrate 520 in the base 512 while contacting only a relatively small area of the first substrate 520.

In operation, bodily fluid (e.g., whole blood) is received in the channel 516 at the upper portion 518a of the base 512 (e.g., after the patient actuates the actuator 104 to deploy the skin-piercing feature from within the device 100, as shown in FIGS. 1A-1C). The blood flows through/along the channel 516 toward the reservoir portion 519 (e.g., in the direction indicated by the arrow C in FIG. 5B), where the blood is absorbed by the second substrate 560. The second substrate 560 then transfers the blood into a first end portion 522 of the first substrate 520, and the first substrate 520 wicks the blood from the first end portion 522 to a second end portion 526 (e.g., in the direction indicated by the arrow D in FIG. 5A), such that plasma is separated and collected farther toward the second end portion 526 of the first substrate 520. In one aspect of the present technology, the second substrate 560 can quickly absorb a large volume of the blood before transferring the blood to the first substrate 520 at a controlled rate. All or a portion of the first substrate 520 can then be removed from the housing 510 (e.g., punched out) to allow for testing of the plasma and/or other blood constituents collected therein.

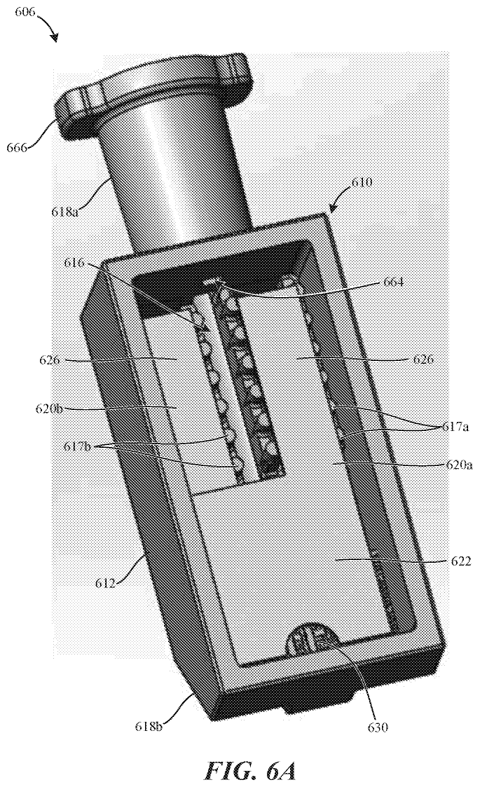

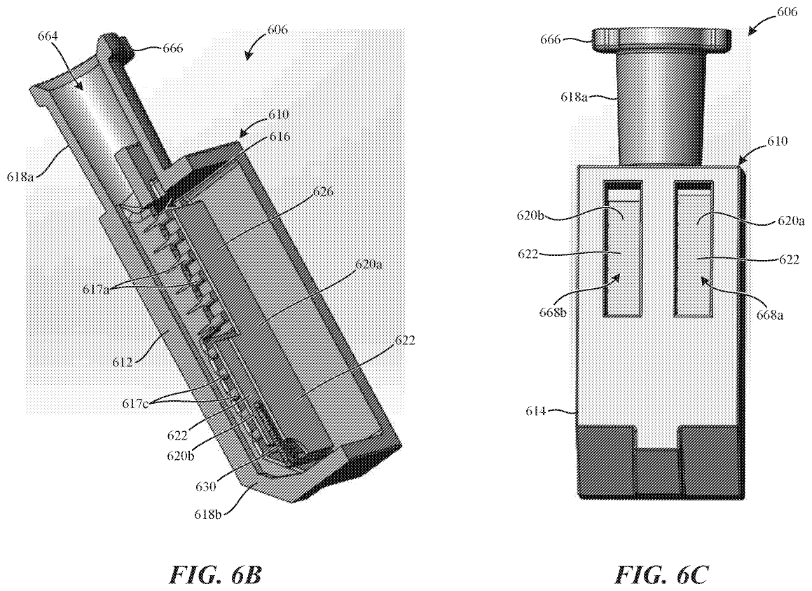

FIGS. 6A-6C are an isometric front view, a cross-sectional side view, and a front view, respectively, of a collection cartridge 606 configured in accordance with another embodiment of the present technology. Referring to FIGS. 6A-6C together, the cartridge 606 can include some features generally similar or identical to the features of the cartridges 106, 406, and/or 506 described in detail above with reference to FIGS. 1A-5B, and can be used with the bodily fluid collection device 100.

In the illustrated embodiment, the cartridge 606 includes a housing 610, a pair of substrates 620 (identified individually as a first substrate 620a and a second substrate 620b), and a divider 630. The housing 610 includes a base 612 having an upper portion 618a and a lower portion 618b, and a cover 614 configured to be (e.g., removably) coupled to the base 612. The cover 614 is not shown in FIGS. 6A and 6B for clarity. The upper portion 618a can define an inflow channel 664 for receiving a flow of bodily fluid (e.g., blood), and can include one or more mating features 666 (e.g., flanges) for releasably coupling the cartridge 606 to the device 100. In the illustrated embodiment, the housing 610 defines a channel 616 extending from the upper portion 618a toward the lower portion 618b of the base 612. The channel 616 is fluidly connected to the inflow channel 664.

The substrates 620 may be composed of any suitable material for receiving, collecting, and/or stabilizing bodily fluid, such as a paper or fiber matrix. In some embodiments, the substrates 620 are both configured to separate plasma from a sample of whole blood. In the illustrated embodiment, the substrates 620 are generally identical and each include (i) a lower portion 622 and (ii) an upper portion 626 that has a smaller dimension (e.g., width, area) than the lower portion 626. Accordingly, the substrates 620 can have a generally “L”-shape. In the illustrated embodiment, the first substrate 620a is (i) positioned over the second substrate 620b within the base 612 and (ii) oriented differently (e.g., flipped) relative the second substrate 620b. Therefore, (i) the upper portions 626 of the substrates 620 can be spaced apart from one another with the channel 616 positioned laterally therebetween, while (ii) the lower portions 622 of the substrates 620 can be positioned over/adjacent to one another. Moreover, in the illustrated embodiment the housing 610 includes a plurality of stand-off members 617 (identified individually as first stand-off members 617a, second stand-off members 617b, and third stand-off members 617c) projecting inward from the base 612 and toward the substrates 620. The first stand-off members 617a can engage the upper portion 626 of the first substrate 620a; the second stand-off members 617b can engage the upper portion 626 of the second substrate 620b; and the third stand-off members 617c can engage the lower portion 622 of the second substrate 620b. In some embodiments, the first and second stand-off members 617a, b can be taller than the third stand-off members 617c. By this arrangement, the stand-off members 617 secure/suspend the substrates 620 within the base 612 of the housing 610.

In the illustrated embodiment, the divider 630 (e.g., a plastic mesh) is positioned between the lower portions 622 of the substrates 620 to maintain separation therebetween. In other embodiments, the divider 630 can be omitted, or the divider 630 can comprise an absorbent substrate (e.g., a paper or fiber matrix) for receiving a bodily fluid. For example, the divider 630 can be a thicker and/or relatively more absorbent substrate than the substrates 620 and/or configured to control a rate of fluid transfer into the substrates 620.

In operation, bodily fluid (e.g., whole blood) is received in the channel 616 via the inflow channel 664 (e.g., after the patient actuates the actuator 104 to deploy the skin-piercing feature from within the device 100, as shown in FIGS. 1A-1C). The blood flows (i) through/along the channel 616 and toward the lower portions 622 of the substrates 620, and (ii) into the space between the lower portions 622 of the substrates 620. The lower portions 622 of the substrates 620 then absorb and wick the blood toward the upper portions 626 thereof such that plasma is separated and collected farther toward the upper portions 626 of the substrates 620. In one aspect of the present technology, the channel 616 is spaced apart from the upper portions 626 of the substrates 620 such that blood is not transferred from the channel 616 into the upper portions 626 of the substrates 620.

In some embodiments, the cover 614 includes a first aperture 668a and a second aperture 668b aligned with the upper portions 626 of the substrates 620, respectively. In some embodiments, the base 612 can include corresponding apertures. In operation, the upper portions 626 of the substrates 620 (e.g., containing separated plasma) can be removed (e.g., punched out) from within the housing 610 via the first and second apertures 668a, b. In one aspect of the present technology, the cartridge 606 enables two separate plasma samples to be obtained for the same sample of whole blood—thereby enabling additional testing on the same sample.

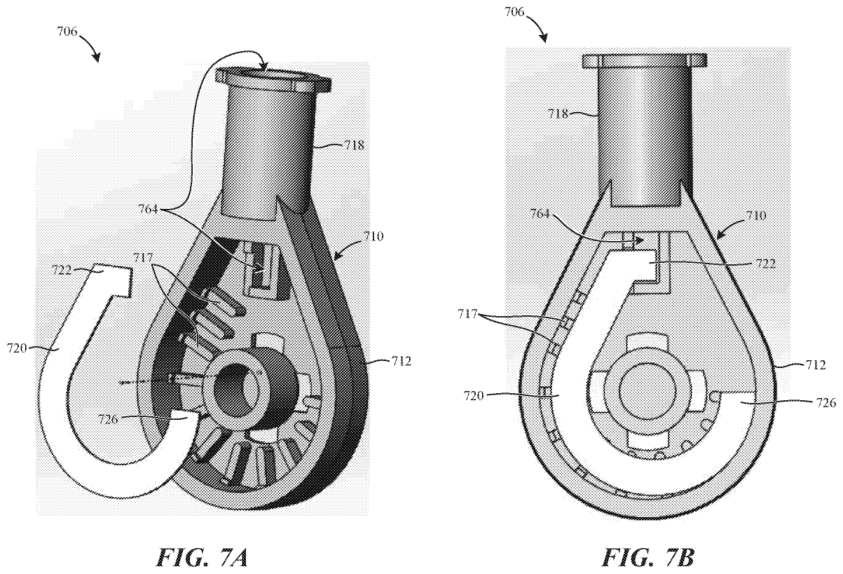

FIGS. 7A and 7B are an exploded isometric view and a front view, respectively, of a collection cartridge 706 configured in accordance with another embodiment of the present technology. Referring to FIGS. 7A and 7B together, the cartridge 706 can include some features generally similar or identical to the features of the cartridges 106, 406, 506, and/or 606 described in detail above with reference to FIGS. 1A-6C, and can be used with the bodily fluid collection device 100.

In the illustrated embodiment, the cartridge 706 includes a housing 710 and a substrate 720 positioned within the housing 710. The housing 710 can include a base 712 and a cover configured to be (e.g., removably) coupled to the base 712. The cover is not shown in FIGS. 7A and 7B for clarity. The base 712 includes a coupling portion 718 configured to be releasably coupled to the housing 102 of the device 100 (FIGS. 1A-1C). The base 712 (e.g., the coupling portion 718) further defines an inflow channel 764 for receiving a flow of bodily fluid (e.g., blood).

The substrate 720 may be composed of any suitable material for receiving, collecting, and/or stabilizing bodily fluid, such as a paper or fiber matrix. In some embodiments, the substrate 720 is configured to separate plasma from a sample of whole blood. The substrate 720 includes a first end portion 722 positioned within the inflow channel 764, and has a generally curved/arcuate shape. In some embodiments, the substrate 720 can be positioned on stand-off members 717 within the base 712 of the housing 710.

In operation, bodily fluid (e.g., whole blood) is received in the inflow channel 764 (e.g., after the patient actuates the actuator 104 to deploy the skin-piercing feature from within the device 100, as shown in FIGS. 1A-1C). The blood then flows from the inflow channel 764 into the first end portion 722 of the substrate 720, and the substrate 720 wicks the blood toward a second end portion 726 thereof such that plasma is separated and collected farther toward the second end portion 726 of the substrate 720. In one aspect of the present technology, the curved shape of the substrate 720 provides effective plasma separation within a small footprint—allowing the housing 710 to be made relatively smaller.

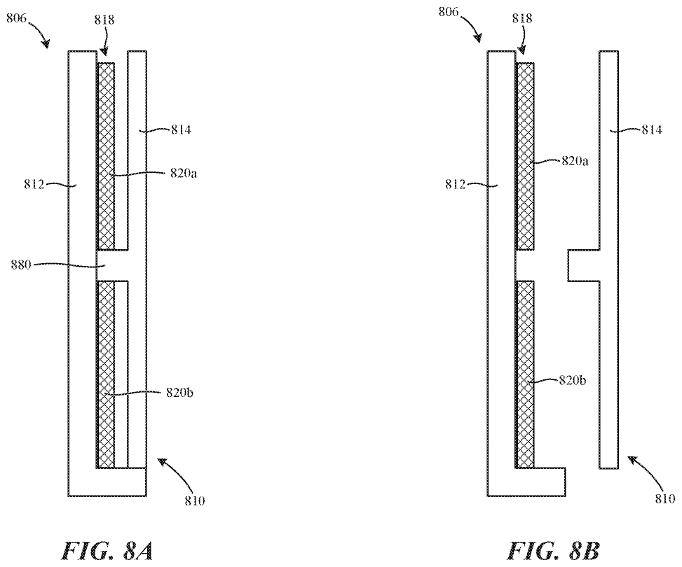

FIGS. 8A and 8B are side cross-sectional views of a collection cartridge 806 in a first position and a second position, respectively, configured in accordance with another embodiment of the present technology. Referring to FIGS. 8A and 8B together, the cartridge 806 can include some features generally similar or identical to the features of the cartridges 106, 406, 506, 606, and/or 706 described in detail above with reference to FIGS. 1A-7B, and can be used with the bodily fluid collection device 100. For example, the cartridge 806 includes (i) a housing 810 having a base 812 and a cover 814 (e.g., an adhesive layer) configured to be releasably coupled to the base 812, and (ii) a pair of substrates 820 (identified individually as a first substrate 820a and a second substrate 820b; e.g., strips of plasma separation paper) positioned within the base 812. The base 812 further includes an upper portion 818 configured to receive a flow of bodily fluid.

In the illustrated embodiment, the cover 814 further includes a bridge portion 880. In some embodiments, the bridge portion 880 comprises a paper or substrate (e.g., a transfer substrate) configured to absorb and transfer fluid. In other embodiments, the bridge portion 880 can alternatively or additionally comprise one or more microfluidic features (e.g., closed microfluidic channels, open microfluidic channels) configured to route fluid. In the first position shown in FIG. 8A, the bridge portion 880 is positioned between the substrates 820 and fluidly connects the substrates 820 such that fluid can flow from the first substrate 820a to the second substrate 820b. In the second position shown in FIG. 8B, the cover 814 is moved (e.g., peeled off) such that the bridge portion 880 no longer spans between and fluidly connects the first substrate 820a and the second substrate 820b. Thus, fluid is inhibited or even prevented from flowing into the second substrate 820b when the cover 814 is in the second position.

In operation, bodily fluid (e.g., whole blood) is received through the upper portion 818 of the base 812 with the cover 814 in the first position. The blood then flows (i) into the first substrate 820a and through/along the first substrate 820a, (ii) from the first substrate 820a through/along the bridge portion 880 and into the second substrate 820b, and (iii) through/along the second substrate 820b. As the blood moves through the substrates 820, plasma is separated and collected primarily in the second substrate 820b. After a suitable time period (e.g., collection period), the cover 814 is moved to the second position (e.g., removed from the base 812) to inhibit additional blood flow to/into the second substrate 820b. In one aspect of the present technology, fluidly disconnecting the substrates 820 by moving the cover 814 to the second position inhibits additional blood from saturating and potentially contaminating the second substrate 820b with blood constituents other than plasma.

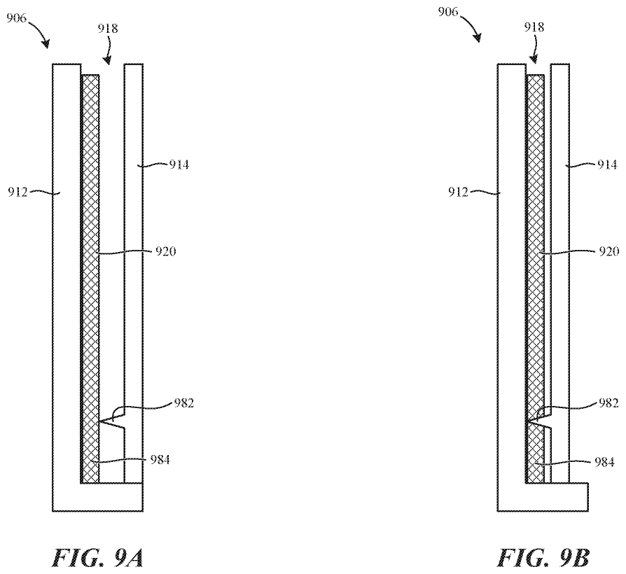

FIGS. 9A and 9B are side cross-sectional views of a collection cartridge 906 in a first position and a second position, respectively, configured in accordance with another embodiment of the present technology. Referring to FIGS. 9A and 9B together, the cartridge 906 can include some features generally similar or identical to the features of the cartridges 106, 406, 506, 606, 706, and/or 806 described in detail above with reference to FIGS. 1A-8B, and can be used with the bodily fluid collection device 100. For example, in the illustrated embodiment the cartridge 906 includes (i) a housing 910 including a base 912 and a cover 914 (e.g., an adhesive layer) configured to be releasably coupled to the base 912, and (ii) a substrate 920 (e.g., a strip of plasma separation paper) positioned within the base 912. The base 912 further includes an upper portion 918 configured to receive a flow of bodily fluid.

In the illustrated embodiment, the cover 914 further includes a cutting mechanism 982. In some embodiments, the cutting mechanism 982 is a sharpened section of the (e.g., plastic) cover 914 while, in other embodiments, the cutting mechanism 982 is a metal blade or other sharpened instrument attached to the cover 914. In the first position shown in FIG. 9A, the cutting mechanism 982 is positioned adjacent to the substrate 920. In the second position shown in FIG. 9B, the cutting mechanism 982 is actuated toward and into the substrate 920 to cut the substrate 920 to, for example, sever a portion 984 of the substrate 920 to inhibit or even prevent fluid from flowing into the severed portion 984 of the substrate

In operation, bodily fluid (e.g., whole blood) is received through the upper portion 918 of the base 912 with the cover 914 in the first position. The blood then flows through/along the substrate 920. As the blood moves through the substrate 920, plasma is separated and collected toward/primarily in the portion 984 of the substrate 920. After a suitable time period, the cover 914 is moved toward the base 912 to sever the portion 984 and inhibit additional blood flow to/into the portion 984. In one aspect of the present technology, fluidly disconnecting the portion 984 of the substrate 920 by moving the cover 914 to the second position inhibits additional blood from saturating and potentially contaminating the portion 984 with blood constituents other than plasma.

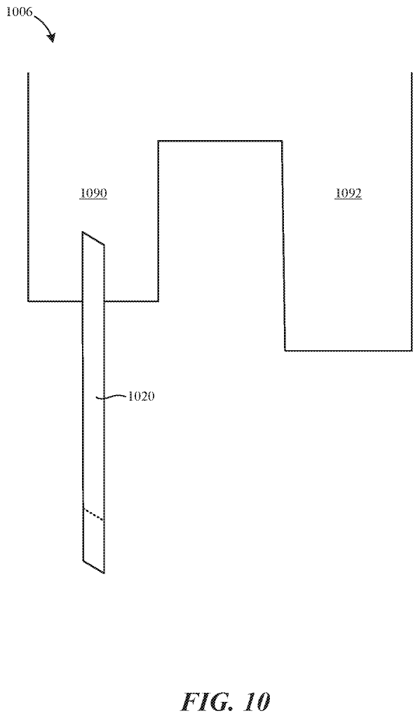

FIG. 10 is a schematic view of a portion of a collection cartridge 1006 configured in accordance with another embodiment of the present technology. In the illustrated embodiment, the cartridge 1006 includes a (i) an inlet reservoir 1090, (ii) a substrate 1020 fluidly connected to the inlet reservoir 1090, and (iii) an overflow reservoir 1092. In some embodiments, the overflow reservoir 1092 can have a greater volume than the inlet reservoir 1090. In operation, bodily fluid (e.g., whole blood) is received in the inlet reservoir 1090 and absorbed by the substrate 1020. If the amount of blood exceeds the volume of the inlet reservoir 1090 (e.g., a flow rate of the received blood exceeds the absorption rate of the substrate 1020), the blood can flow into the overflow reservoir 1092 to inhibit excess saturation and potential contamination of the substrate 1020. As one of ordinary skill in the art will appreciate, any of the collection cartridges described in detail herein can include one or more overflow reservoirs.

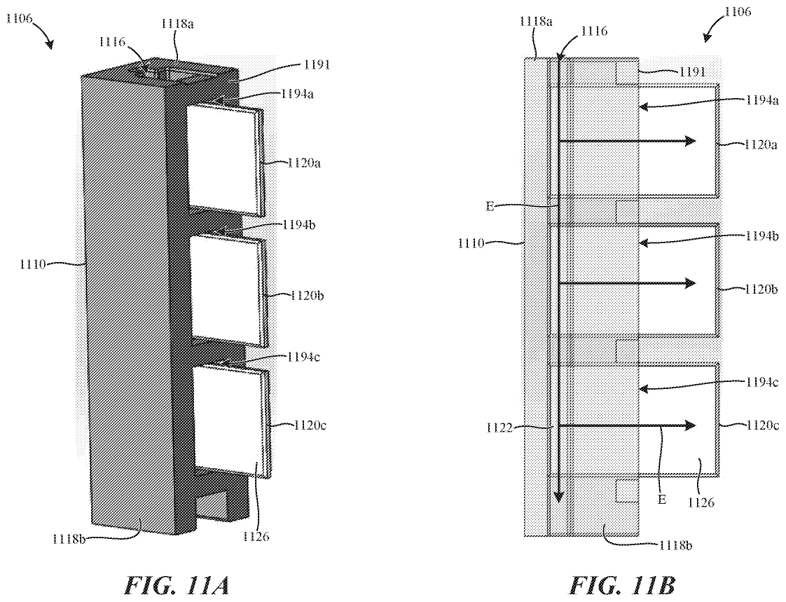

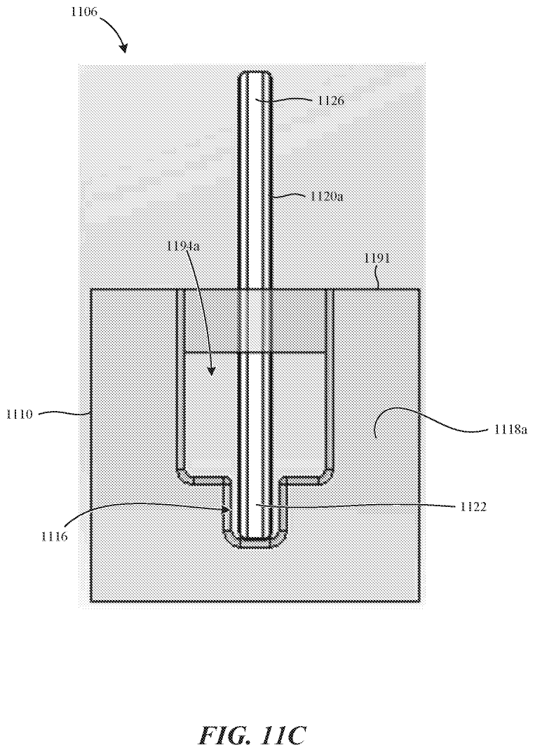

FIGS. 11A-11C are an isometric view, a side view, and a top view, respectively, of a collection cartridge 1106 configured in accordance with another embodiment of the present technology. Referring to FIGS. 11A-11C together, the cartridge 1106 can include some features generally similar or identical to the features of the cartridges 106, 406, 506, 606, 706, 806, 906, and/or 1006 described in detail above with reference to FIGS. 1A-10, and can be used with the bodily fluid collection device 100 and/or other suitable bodily fluid collection devices.

In the illustrated embodiment, the cartridge 1106 includes a housing 1110 and a plurality of substrates 1120 (identified individually as a first substrate 1120a, a second substrate 1120b, and a third substrate 1120c). The housing 1110 is shown as transparent in FIGS. 11B and 11C for clarity. The housing 1110 includes an upper portion 1118a (e.g., a first portion, a first end portion, an upper end portion) and a lower portion 1118b (e.g., a second portion, a second end portion, an upper end portion), and defines a channel 1116 extending from the upper portion 1118a at least partially toward the lower portion 1118b. In some embodiments, the housing 1110 can include or be positioned within a cover and/or a base (not shown) that can be removably coupled together. In some embodiments, the upper portion 1118a is configured to be removably coupled to the device 100 (FIGS. 1A-1C) and, in some embodiments, can include one or more mating features (e.g., flanges), connectors, tubes, and/or other features (not shown) for coupling the cartridge 1106 to the device 100. In the illustrated embodiment, the housing 1110 further includes a plurality of openings 1194 (identified individually as a first opening 1194a, a second opening 1194b, and a third opening 1194c) extending through a sidewall 1191 of the housing 110 and over the channel 1116.

In the illustrated embodiment, the substrates 1120 are each positioned in corresponding ones of the openings 1194. More specifically, each of the substrates 1120 can include (i) a first end portion 1122 positioned within the housing 1110 and in fluid communication with the channel 1116 (e.g., positioned within the channel 1116) and (ii) a second end portion 1126 positioned outside the housing 1110. In the illustrated embodiment, the substrates 1120 have identical dimensions and arrangements relative to the openings 1194 while, in other embodiments, the substrates 1120 can have varying dimensions, varying arrangements, and/or a different number (e.g., more or fewer than the three illustrated substrates 1120). The substrates 1120 may be composed of any suitable material for receiving, collecting, and/or stabilizing bodily fluid, such as a paper or fiber matrix. In some embodiments, each of the substrates 1120 can be configured to separate plasma from whole blood.

In operation, bodily fluid (e.g., whole blood) is received in the channel 1116 at the upper portion 1118a of the housing 1110 (e.g., after the patient actuates the actuator 104 to deploy the skin-piercing feature from within the device 100, as shown in FIGS. 1A-1C). The blood initially flows through/along the channel 1116 toward the first substrate 1120a where it is absorbed into the first end portion 1122 of the first substrate 1120a. The first substrate 1120a wicks the blood from the first end portion 1122 toward the second end portion 1126, such that plasma is separated and collected farther toward the second end portion 1126 of the first substrate 1120a. In some embodiments, the blood begins to flow through the channel 1116 toward the second substrate 1120b only after the first substrate 1120a is sufficiently saturated. In other embodiments, the blood begins to flow through the channel 1116 toward the second substrate 1120b while the first substrate 1120a is simultaneously absorbing and wicking blood. In either arrangement, the first end portion 1122 of the second substrate 1120b begins to absorb blood after the first substrate 1120a and can similarly wick the blood toward the second end portion 1126 thereof to separate and collect plasma toward the second end portion 1126. The flow of blood can proceed in this sequential manner to the third substrate 1120c (and any additional substrates) which can similarly absorb and separate plasma. The overall direction of blood flow through the cartridge 1106 is indicated by the series of arrows E shown in FIG. 11B.

Accordingly, in one aspect of the present technology the cartridge 1106 is configured to facilitate sequential filling of the substrates 1120 as the blood progresses through the channel 1116 and wicks outward along the substrates 1120. After some or all of the substrates 1120 are filled, the substrates 1120 can be removed from the housing 1110 to allow for testing of the plasma and/or other blood constituents collected therein. In another aspect of the present technology, the cartridge 1106 enables multiple plasma samples to be obtained for the same sample of whole blood as each of the substrates 1120 can be used for testing individually—thereby enabling additional testing on the same sample. In some embodiments, the cartridge 1106 can further include an overflow reservoir (e.g., an absorbent pad; not shown) positioned near the lower portion 1118b of the housing 1110 and in fluid communication with the channel 1116 for receiving and absorbing excess blood and/or other bodily fluids.

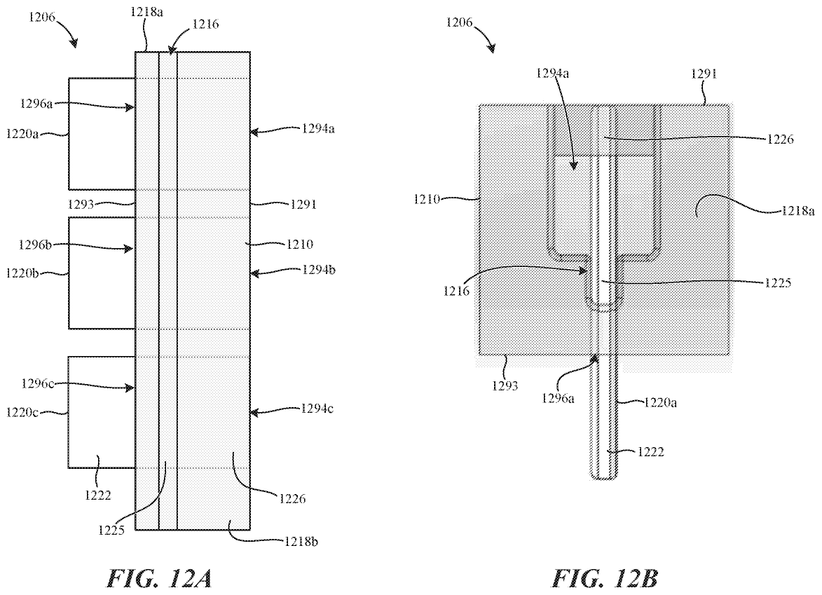

FIGS. 12A and 12B are a side view and a top view, respectively, of a collection cartridge 1206 configured in accordance with another embodiment of the present technology. Referring to FIGS. 12A and 12B together, the cartridge 1206 can include some features generally similar or identical to the features of the cartridge 1106 described in detail above with reference to FIGS. 11A-11C, and can be used with the bodily fluid collection device 100 and/or other suitable bodily fluid collection devices. For example, in the illustrated embodiment the cartridge 1206 includes a housing 1210 including an upper portion 1218a and a lower portion 1218b, and defining a channel 1216 extending at least partially from the upper portion 1218a toward the lower portion 1218b. The housing 1210 is shown as transparent in FIGS. 12A and 12B for clarity. The housing 1210 further includes (i) a plurality of openings 1294 (identified individually as a first opening 1294a, a second opening 1294b, and a third opening 1294c) extending through a first sidewall 1291 of the housing 1210 and over the channel 1216 and (ii) a plurality of substrates 1220 (identified individually as a first substrate 1220a, a second substrate 1220b, and a third substrate 1220c).

In the illustrated embodiment, the housing 1210 also includes a plurality of slots 1296 (identified individually as a first slot 1296a, a second slot 1296b, and a third slot 1296c) that extend through a second sidewall 1293 of the housing 1210 over the channel 1216. The slots 1296 can be aligned with corresponding ones of the openings 1294. The openings 1294 and the slots 1296 can each or individually be referred to as slots, openings, apertures, gaps, and the like. In the illustrated embodiment, the substrates 1220 are each positioned in corresponding ones of the openings 1294 and the slots 1296. More specifically, each of the substrates 1220 can include (i) a first end portion 1222 positioned outside the housing 1210 adjacent the corresponding one of the slots 1296, (ii) a middle portion 1225 positioned within the channel 1216, and (iii) a second end portion 1226 positioned within the corresponding one of the openings 1294. In the illustrated embodiment, the substrates 1220 extend beyond the second sidewall 1293 of the housing 1210 but do not extend beyond the first sidewall 1291 of the housing 1210. In other embodiments, some or all of the substrates 1220 can extend beyond both the first sidewall 1291 and the second sidewall 1293, beyond only the first sidewall 1291, or beyond neither the first sidewall 1291 nor the second sidewall 1293 (e.g., the substrates 1220 can be contained entirely within the housing 1210).

The substrates 1220 may be composed of any suitable material for receiving, collecting, and/or stabilizing bodily fluid, such as a paper or fiber matrix. In some embodiments, each of the substrates 1220 can be configured to separate plasma from whole blood. In operation, bodily fluid (e.g., whole blood) is received in the channel 1216 at the upper portion 1218a of the housing 1210 (e.g., after the patient actuates the actuator 104 to deploy the skin-piercing feature from within the device 100, as shown in FIGS. 1A-1C). The blood initially flows through/along the channel 1216 toward the first substrate 1220a where it is absorbed into the middle portion 1225 of the first substrate 1220a. The first substrate 1220a wicks the blood from the middle portion 1225 toward the first end portion 1222 and toward the second end portion 1226 such that plasma is separated and collected farther toward the first and second end portions 1222, 1226 of the first substrate 1220a. In some embodiments, blood begins to flow through the channel 1216 toward the second substrate 1220b only after the first substrate 1220a is sufficiently saturated. In other embodiments, blood begins to flow through the channel 1216 toward the second substrate 1220b while the first substrate 1220 is simultaneously absorbing and wicking blood. In either arrangement, the middle portion 1225 of the second substrate 1220b begins to absorb blood after the first substrate 1220a and can similarly wick the blood toward the first and second end portions 1222, 1226 thereof to separate and collect plasma toward the first and second end portions 1222, 1226. The flow of blood can proceed in this sequential manner to the third substrate 1220c (and any additional substrates) which can similarly absorb and separate plasma from the blood.

Accordingly, in one aspect of the present technology the cartridge 1206 is configured to facilitate sequential filling of the substrates 1220 as the blood progresses through the channel 1216 and wicks outward along the substrates 1220. After some or all of the substrates 1220 are filled, the substrates 1220 can be removed from the housing 1210 to allow for testing of the plasma and/or other blood constituents collected therein. In another aspect of the present technology, the cartridge 1206 enables more plasma samples to be obtained for the same sample of whole blood as compared to the cartridge 1106 described in detail with reference to FIGS. 11A-11C, as plasma is isolated in both of the first and second end portions 1222, 1226 of the substrates 1220—thereby enabling additional testing on the same sample. In some embodiments, the cartridge 1206 can further include an overflow reservoir (e.g., an absorbent pad; not shown) positioned near the lower portion 1218b of the housing 1210 and in fluid communication with the channel 1216 for receiving and absorbing excess blood.

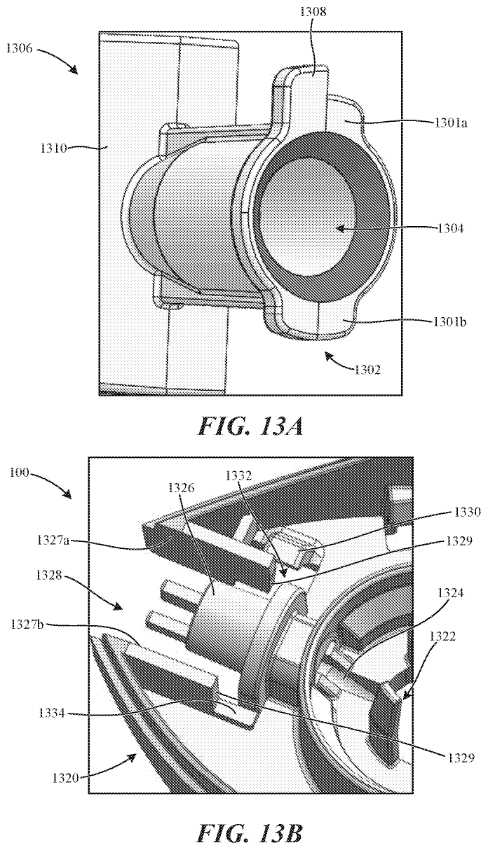

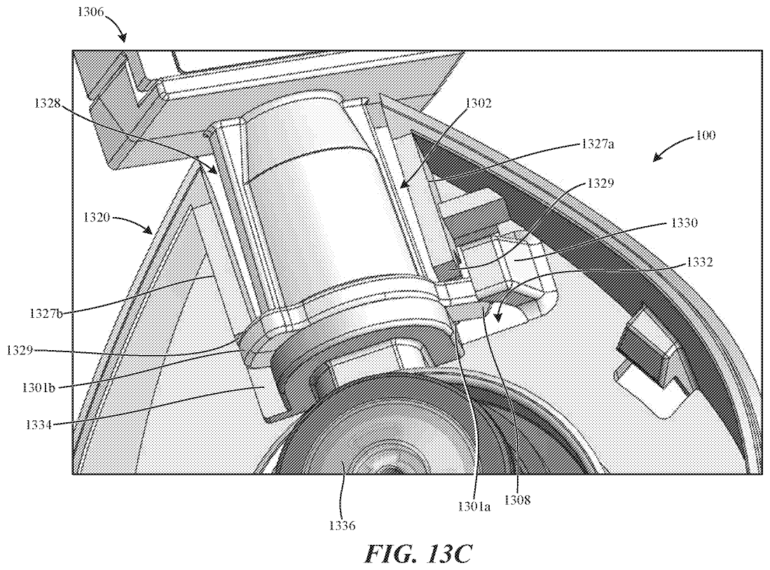

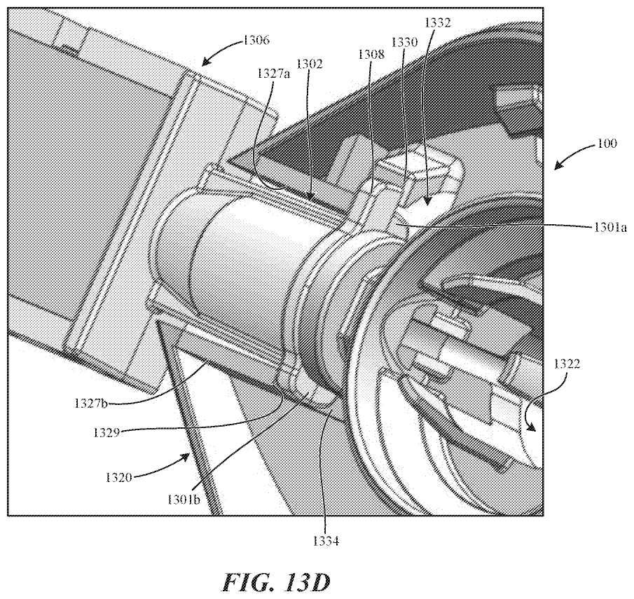

FIG. 13A is an enlarged isometric view of a collection cartridge 1306 configured in accordance with an embodiment of the present technology. FIG. 13B is an enlarged isometric view of an interior portion of the device 100 shown in FIGS. 1A-1C configured in accordance with an embodiment of the present technology. FIGS. 13C and 13D are enlarged isometric view of the collection cartridge 1306 coupled to the portion of the device 100 shown in FIG. 13B in a first position and a second position, respectively, in accordance with embodiments of the present technology.

Referring first to FIG. 13A, the collection cartridge 1306 includes a coupling portion 1302 extending from a housing 1310. One or more substrates (e.g., plasma separation substrates) can be positioned within the housing 1310 in any one or combination of the arrangements described in detail above with reference to FIGS. 2A-12B. In the illustrated embodiment, the coupling portion 1302 defines a fluid inlet 1304 and includes a pair of flanges 1301 (identified individually as a first flange 1301a and a second flange 1301b). A locking feature 1308 extends/projects (e.g., eccentrically) from the first flange 1301a.

Referring next to FIG. 13B, the device 100 can include a base 1320 having (i) a fluid collection site 1322, (ii) a fluidic channel 1324 (e.g., a sloped open microfluidic channel) fluidly connected to and extending away from the collection site 1322, and (iii) an outflow channel 1326 fluidly connected to and extending away from the fluidic channel 1324. In the illustrated embodiment, the base 1320 further includes a pair of sidewalls 1327 (identified individually as a first sidewall 1327a and a second sidewall 1327b) each having an end surface 1329 and collectively defining an opening/slot 1328 that is aligned with the outflow channel 1326. In the illustrated embodiment, the base 1320 further includes (i) a locking feature 1330 positioned adjacent to the end surface 1329 of the first sidewall 1327a and over an opening 1332 in the base 1320 and (ii) a stop member/surface 1334 positioned adjacent to the end surface 1329 of the second sidewall 1327b. The locking feature 1330 can be a flexible tab, projection, hook, and/or other feature configured to engage the locking feature 1308 of the cartridge 1306 as shown in FIG. 13D to lock the cartridge 1306 to the device 100. In some embodiments, the device 100 can include one or more features that are generally similar to or identical to the bodily fluid collection devices disclosed in (i) U.S. patent application Ser. No. 14/816,994, titled “DEVICES, SYSTEMS AND METHODS FOR GRAVITY-ENHANCED MICROFLUIDIC COLLECTION, HANDLING AND TRANSFERRING OF FLUIDS,” filed Aug. 3, 2015; and/or (ii) U.S. patent application Ser. No. 15/711,746, titled “METHODS FOR DELIVERY OF BODILY FLUIDS ONTO A FIBROUS SUBSTRATE,” filed Sep. 21, 2017, each of which is incorporated herein by reference in its entirety.

Referring to FIG. 13C, the coupling portion 1302 of the cartridge 1306 can be inserted into the opening 1328 in the base 1320 of the device 100 with (i) the flanges 1301 oriented generally vertically relative to an axis extending between/through the sidewalls 1327 and with (ii) the first flange 1301a and the locking feature 1308 of the cartridge 1306 positioned below the second flange 1301b. As the cartridge 1306 is inserted into the opening 1328, the fluid inlet 1304 of the cartridge 1306 receives the outflow channel 1326 of the device 100, thereby fluidly connecting the cartridge 1306 to the device 100 and the fluid collection site 1322 (obscured by a membrane and plunger assembly 1336 in FIG. 13C). The cartridge 1306 can then be rotated (e.g., counterclockwise) such that the locking feature 1308 of the cartridge 1306 rotates through the opening 1332 in the base 1320 until the locking feature 1308 engages a lower surface of the locking feature 1330.

Referring next to FIG. 13D, continued rotation of the cartridge 1306 causes the locking feature 1330 of the base 1320 to flex/move until the locking feature 1308 of the cartridge 1306 moves upward past (e.g., snaps by) the locking feature 1330. Once the locking feature 1308 of the cartridge 1306 moves past the locking feature 1330 of the base 1320, the locking feature 1330 can (e.g., elastically) return to its original position (FIGS. 13B and 13C) such that the locking feature 1330 inhibits/blocks the cartridge 1306 from being rotated in the opposite direction (e.g., clockwise). Moreover, in the illustrated embodiment (i) the flanges 1301 are secured behind/against the end surfaces 1329 of the sidewalls 1327, and (ii) the second flange 1301b engages the stop member 1334 of the base 1320 to inhibit/block continued (e.g., counterclockwise) rotation of the cartridge 1306. By this arrangement, the cartridge 1306 is securely locked to the device 100.

In one aspect of the present technology, the cartridge 1306 can be locked to the device 100 during and after use of the device 100 (e.g., to collect whole blood within the cartridge 1306). In some such embodiments, the cartridge 1306 and the device 100 can be shipped together in the locked configuration to a testing facility. For example, the device 100 may include electronics or other components that facilitate maintenance, testing, and/or analysis of the fluid sample collected within the cartridge 1306 during transit to the testing facility.

The following examples are illustrative of several embodiments of the present technology:

1. A device for separating plasma from whole blood, the device comprising:

a housing including a reservoir portion configured to receive the whole blood; and

a plasma separation substrate positioned at least partially within the housing,
wherein the plasma separation substrate is folded to define (a) a crease, (b) a first strip portion extending away from the crease, and (c) a second strip portion extending away from the crease,
wherein the crease is positioned adjacent to the reservoir portion to receive the whole blood, and
wherein the plasma separation substrate is configured to wick the whole blood along the first and second strip portions to separate the plasma from the whole blood.

2. The device of example 1, further comprising a divider positioned between the first and second strip portions, wherein the divider is configured to inhibit fluid flow between the first and second strip portions.

3. The device of example 2 wherein the divider comprises a plastic mesh.

4. The device of any one of examples 1-3 wherein the housing defines a channel and includes a plurality of stand-off members configured to support the plasma separation substrate within the channel.

5. The device of any one of examples 1-4, further comprising an overflow substrate positioned within the housing and configured to receive a volume of whole blood exceeding a saturation volume of the plasma separation substrate.

6. The device of any of examples 1-5, further comprising a button operably coupled to a substrate-piercing feature, wherein the button is actuatable to separate a portion of the plasma separation substrate containing at least a portion of the plasma.

7. The device of any one of examples 1-6 wherein the housing includes a cover and a base, wherein the cover is removable from the base to permit removal of the plasma separation substrate.

8. The device of any one of examples 1-7 wherein the first strip portion extending away from the crease is generally parallel to the second strip portion extending away from the crease.

9. A device for separating plasma from whole blood, the device comprising:
a housing including a channel configured to receive the whole blood;
a transfer substrate positioned at least partially within the housing and fluidly connected to the channel; and
a plasma separation substrate positioned at least partially within the housing and fluidly connected to the transfer substrate,
   wherein the transfer substrate is configured to absorb the whole blood from the channel and transfer the whole blood to the plasma separation substrate, and
   wherein the plasma separation substrate is configured to wick the whole blood along a length thereof to separate the plasma from the whole blood.

10. The device of example 9 wherein the transfer substrate has a greater rate of absorption than the plasma separation substrate.

11. The device of example 9 or example 10 wherein the housing further includes a coupling portion configured to be removably coupled to a fluid collection device for receiving the whole blood, wherein the coupling portion defines the channel, and wherein the transfer substrate is positioned adjacent the coupling portion of the housing.

12. The device of any one of examples 9-11 wherein the housing further includes (a) a coupling portion configured to be removably coupled to a fluid collection device for receiving the whole blood and (b) a base portion opposite the coupling portion, wherein the channel extends along a length of the housing between the coupling portion and the base portion, and wherein the transfer substrate is positioned adjacent the base portion.

13. The device of any one of examples 9-12 wherein the housing includes a base and a cover removably coupled to the base, and wherein the transfer substrate is positioned between the plasma separation substrate and the base in a direction extending between the cover and the base.

14. The device of any one of examples 9-13 wherein the plasma separation substrate is a first plasma separation substrate, and further comprising a second plasma separation substrate positioned at least partially within the housing and fluidly connected to the transfer substrate, wherein the first and second plasma separation substrates each have a generally L-shape.

15. A device for separating plasma from whole blood, the device comprising:
a housing having a first portion and a second portion opposite the first portion, wherein the housing includes a channel extending from the first portion at least partially toward the second portion, and wherein the channel is configured to receive the whole blood; and
a plurality of plasma separation substrates supported by the housing and fluidly connected to the channel, wherein the plasma separation substrates are (a) spaced apart along the housing between the first and second portions to sequentially absorb a sequentially-received portion of the whole blood and (b) configured to separate the plasma from the sequentially-received portion of the whole blood.

16. The device of example 15 wherein the housing includes a sidewall extending between the first and second portions, wherein the sidewall includes a plurality of openings therein, and wherein individual ones of the plasma separation substrates extend from within the housing at least partially through a corresponding one of the openings.

17. The device of example 16 wherein the individual ones of the plasma separation substrates include (a) a first end portion positioned within the channel within the housing and (b) a second portion extending through the corresponding one of the openings outside the housing, and wherein the individual ones of the plasma separation substrates are configured to wick the sequentially-received portion of the whole blood from the first end portion toward the second portion to separate the plasma.

18. The device of any one of examples 15-17 wherein the housing includes a first sidewall and a second sidewall extending between the first and second portions, wherein the first sidewall includes a plurality of first openings therein, wherein the second sidewall includes a plurality of second openings therein, wherein the first openings are aligned with corresponding ones of the second openings, wherein the channel extends between the first and second openings, and wherein individual ones of the plasma separation substrates extend from within the housing at least partially through a corresponding one of the first openings and a corresponding one of the second openings.

19. The device of example 18 wherein the individual ones of the plasma separation substrates include (a) a first end portion extending past the corresponding one of the first openings outside the housing, (b) a middle portion positioned within the channel within the housing, and (c) a second portion extending past the corresponding one of the second openings outside the housing, and wherein the individual ones of the plasma separation substrates are configured to wick the sequentially-received portion of the whole blood from the middle portion toward both the first end portion and the second end portion.

20. The device of any one of examples 15-19 wherein the plasma separation substrates have a rectangular shape, and wherein the plurality of plasma separation substrates includes three plasma separation substrates.

The above detailed description of embodiments of the technology are not intended to be exhaustive or to limit the technology to the precise form disclosed above. Although specific embodiments of, and examples for, the technology are described above for illustrative purposes, various equivalent modifications are possible within the scope of the technology as those skilled in the relevant art will recognize. For example, although steps are presented in a given order, alternative embodiments may perform steps in a different order. The various embodiments described herein may also be combined to provide further embodiments.

From the foregoing, it will be appreciated that specific embodiments of the technology have been described herein for purposes of illustration, but well-known structures and functions have not been shown or described in detail to avoid unnecessarily obscuring the description of the embodiments of the technology. Where the context permits, singular or plural terms may also include the plural or singular term, respectively.

Moreover, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. Additionally, the term “comprising” is used throughout to mean including at least the recited feature(s) such that any greater number of the same feature and/or additional types of other features are not precluded. It will also be appreciated that specific embodiments have been described herein for purposes of illustration, but that various modifications may be made without deviating from the technology. Further, while advantages associated with some embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

I/We claim:

1. A device for separating plasma from whole blood, the device comprising:
a housing including a reservoir portion configured to receive the whole blood; and
a plasma separation substrate positioned at least partially within the housing,
wherein the plasma separation substrate is folded to define (a) a crease, (b) a first strip portion extending away from the crease, and (c) a second strip portion extending away from the crease,
wherein the crease is positioned adjacent to the reservoir portion to receive the whole blood, and
wherein the plasma separation substrate is configured to wick the whole blood along the first and second strip portions to separate the plasma from the whole blood.

2. The device of claim 1, further comprising a divider positioned between the first and second strip portions, wherein the divider is configured to inhibit fluid flow between the first and second strip portions.

3. The device of claim 2 wherein the divider comprises a plastic mesh.

4. The device of claim 1 wherein the housing defines a channel and includes a plurality of stand-off members configured to support the plasma separation substrate within the channel.

5. The device of claim 1, further comprising an overflow substrate positioned within the housing and configured to receive a volume of whole blood exceeding a saturation volume of the plasma separation substrate.

6. The device of claim 1, further comprising a button operably coupled to a substrate-piercing feature, wherein the button is actuatable to separate a portion of the plasma separation substrate containing at least a portion of the plasma.

7. The device of claim 1 wherein the housing includes a cover and a base, wherein the cover is removable from the base to permit removal of the plasma separation substrate.

8. The device of claim 1 wherein the first strip portion extending away from the crease is generally parallel to the second strip portion extending away from the crease.

9. A device for separating plasma from whole blood, the device comprising:
a housing including a channel configured to receive the whole blood;
a transfer substrate positioned at least partially within the housing and fluidly connected to the channel; and
a plasma separation substrate positioned at least partially within the housing and fluidly connected to the transfer substrate,
wherein the transfer substrate is configured to absorb the whole blood from the channel and transfer the whole blood to the plasma separation substrate, and
wherein the plasma separation substrate is configured to wick the whole blood along a length thereof to separate the plasma from the whole blood.

10. The device of claim 9 wherein the transfer substrate has a greater rate of absorption than the plasma separation substrate.

11. The device of claim 9 wherein the housing further includes a coupling portion configured to be removably coupled to a fluid collection device for receiving the whole blood, wherein the coupling portion defines the channel, and wherein the transfer substrate is positioned adjacent the coupling portion of the housing.

12. The device of claim 9 wherein the housing further includes (a) a coupling portion configured to be removably coupled to a fluid collection device for receiving the whole blood and (b) a base portion opposite the coupling portion, wherein the channel extends along a length of the housing between the coupling portion and the base portion, and wherein the transfer substrate is positioned adjacent the base portion.

13. The device of claim 9 wherein the housing includes a base and a cover removably coupled to the base, and wherein the transfer substrate is positioned between the plasma separation substrate and the base in a direction extending between the cover and the base.

14. The device of claim 9 wherein the plasma separation substrate is a first plasma separation substrate, and further comprising a second plasma separation substrate positioned at least partially within the housing and fluidly connected to the transfer substrate, wherein the first and second plasma separation substrates each have a generally L-shape.

15. A device for separating plasma from whole blood, the device comprising:
a housing having a first portion and a second portion opposite the first portion, wherein the housing includes a channel extending from the first portion at least partially toward the second portion, and wherein the channel is configured to receive the whole blood; and
a plurality of plasma separation substrates supported by the housing and fluidly connected to the channel, wherein the plasma separation substrates are (a) spaced apart along the housing between the first and second portions to sequentially absorb a sequentially-received portion of the whole blood and (b) configured to separate the plasma from the sequentially-received portion of the whole blood.

16. The device of claim 15 wherein the housing includes a sidewall extending between the first and second portions, wherein the sidewall includes a plurality of openings therein, and wherein individual ones of the plasma separation substrates extend from within the housing at least partially through a corresponding one of the openings.

17. The device of claim 16 wherein the individual ones of the plasma separation substrates include (a) a first end portion positioned within the channel within the housing and (b) a second portion extending through the corresponding one of the openings outside the housing, and wherein the individual ones of the plasma separation substrates are configured to wick the sequentially-received portion of the whole blood from the first end portion toward the second portion to separate the plasma.

18. The device of claim 15 wherein the housing includes a first sidewall and a second sidewall extending between the first and second portions, wherein the first sidewall includes a plurality of first openings therein, wherein the second sidewall includes a plurality of second openings therein, wherein the first openings are aligned with corresponding ones of the second openings, wherein the channel extends between the first and second openings, and wherein individual ones of the plasma separation substrates extend from within the housing at least partially through a corresponding one of the first openings and a corresponding one of the second openings.

19. The device of claim 18 wherein the individual ones of the plasma separation substrates include (a) a first end portion extending past the corresponding one of the first openings outside the housing, (b) a middle portion positioned within the channel within the housing, and (c) a second portion extending past the corresponding one of the second openings outside the housing, and wherein the individual ones of the plasma separation substrates are configured to wick the sequentially-received portion of the whole blood from the middle portion toward both the first end portion and the second end portion.

20. The device of claim 15 wherein the plasma separation substrates have a rectangular shape, and wherein the plurality of plasma separation substrates includes three plasma separation substrates.
